(19) United States
(12) Patent Application Publication
(10) Pub. No.: US 2022/0280815 A1
(43) Pub. Date: Sep. 8, 2022

(54) SYSTEM AND METHODS FOR OPTICAL IMAGING OF DOSE DEPOSITED BY THERAPEUTIC PROTON BEAMS

(71) Applicants: THE TRUSTEES OF DARTMOUTH COLLEGE, Hanover, NH (US); DOSEOPTICS, LLC, Lebanon, NH (US)

(72) Inventors: Petr BRUZA, Lebanon, NH (US); Brian POGUE, Hanover, NH (US); Michael JERMYN, Lebanon, NH (US); Venkataramanan KRISHNASWAMY, Lebanon, NH (US)

(21) Appl. No.: 17/626,488

(22) PCT Filed: Jul. 10, 2020
(86) PCT No.: PCT/US2020/041683

§ 371 Date: Jan. 11, 2022

Related U.S. Application Data
(60) Provisional application No. 62/873,155, filed on Jul. 11, 2019.

(51) Int. Cl. A61N 5/10; G01T 1/16; G01T 1/29

(52) U.S. Cl. CPC A61N 5/1071; G01T 1/1603; G01T 1/2921; A61N 2005/1059; A61N 2005/1087

(57) ABSTRACT

A system for performing radiation treatment of a patient with a proton beam from a particle accelerator uses a high-sensitivity camera to capture dose images of patient surface, a video processor that integrates the dose images, beam-on detection apparatus, and apparatus to eliminate interference of room lighting. In embodiments, the system registers dose images to a surface model of the patient derived from stereo image pairs captured by a stereo camera. In embodiments, the surface model is registered to three-dimensional images of the patient from MRI or CT, and an integrated three-dimensional energy deposition map of the patient is prepared.

### CROSS-REFERENCE TO RELATED APPLICATIONS

The present application claims priority to U.S. Provisional Patent Application No. 62/873,155 filed 11 Jul. 2019. The entire contents of the aforementioned provisional patent application are incorporated herein by reference.

### GOVERNMENT RIGHTS

This invention was made with government support under grant nos. R01 EB023909 and R44 CA232879 awarded by the National Institutes of Health. The government has certain rights in the invention.

### FIELD

The present document relates to the field of radiation treatment of cancer with high-energy beams of positively-charged subatomic particles, and in particular with proton beams.

### BACKGROUND

When treating cancer with ionizing radiation, it is desirable to maintain a high ratio of radiation energy deposited in the tumor to radiation energy deposited in normal tissues; this ratio is known herein as therapeutic ratio. High energy charged-particle beams, and in particular, proton beams, are sometimes used for such radiation treatments because, with certain beam energies, they can achieve high therapeutic ratio by depositing more energy into subsurface structures where tumor is located than into skin and other overlying tissues. High therapeutic ratio is desirable because with high therapeutic ratio the tumor can receive higher, more lethal, radiation doses while allowing survival of normal tissues.

Normal tissues particularly susceptible to radiation injury during radiation treatment include blood-forming organs such as liver, spleen, and bone marrow, the intestines, and the skin; radiation damage can suppress blood-cell formation, cause sunburn-like inflammation to, or destroy, skin, and impair gut integrity. Proton beams used in treatment are typically tightly focused “pencil” beams, and can often be aimed to avoid enough blood-forming organs to avoid suppression of blood formation even if part of one of the blood-forming organs must be targeted. Skin, however, is penetrated by therapeutic proton beams overlying tumors and may suffer extensive local damage if beams are not directed at the tumor from enough different angles (and thus expose more skin at lower levels) or are too intense.

Proton beams can be swept or steered over limited angles by electromagnets, beams may be steered to intersect with skin, patient, and tumor in a pattern intended to expose an entire tumor to the beams. Further, patients may be positioned in movable chairs or movable beds and their position may be altered during radiation treatment. For example, a patient may be rotated while subjected to a high-energy proton beam to spread skin dose across several different areas of skin while continuing to focus the proton beam on a same tumor in the patient.

Tissues exposed to high-energy electron beams have been imaged under Cherenkov light; Cherenkov light is light emitted as charged particles traveling faster than the phase velocity of light, which is slower in tissue due to tissue's refractive index that is higher than 1. Direct Cherenkov imaging is typically not performed on proton beams because Cherenkov emissions from protons decelerating in water or tissue require 450 MeV beam energies—and most radiation treatment involves beam energies below 450 MeV.

Therapeutic proton beams deposit their energy to patient's tissue mainly via ionizations, multiple Coulomb scattering, and non-elastic nuclear reactions. During proton-beam dose deposition, secondary particles consisting of energetic electrons, protons, neutrons, and to smaller extent X-ray photons, are generated along the primary beam as it interacts with tissue. The energy of both primary protons and secondary electrons in proton therapy is typically below the Cherenkov emission thresholds of about 450 MeV for protons and about 250 keV for electrons.

Therefore, a consensus of those skilled in the art was that the optical yield in tissue exposed to proton beams with beams of less than 450 MeV is too low to be practical for real-time imaging of beam-patient tissue interactions; for example Glaser et al. states “Although emission is present, it is inherently lower than that of x-ray photons and electrons. The weak nature of this emission can be explained by considering direct emission of protons themselves, emission from secondary scattered electrons, and emission from induced radioisotopes. Due to the mass of a proton, per (3) the threshold energy for direct Cherenkov light emission from protons themselves is approximately 485 MeV in water (n=1.33) and 268 MeV in plastic (n=1.59). Given the clinical energy range of proton beams (below 250 MeV), this is not feasible.” (Optical dosimetry of radiotherapy beams using Cherenkov radiation: the relationship between light emission and dose, Adam K Glaser et al., Phys. Med. Biol. 59 3789, 2014). It is known, however, that some light is emitted when proton beams interact with water—a cooled camera with two second exposure times has detected optical signals from a tissue phantom corresponding to a dose deposition profile of a pristine proton beam when camera and tissue phantom are shielded fully from extraneous light (Luminescence imaging of water during proton-beam irradiation for range estimation, Yamamoto, S, Toshito, T, Okumura, S and Komori, M, Med. Phys. 42 (11), November 2015.)

It is believed that these optical signals result from a combination of a radiative pathway of excited state electrons, which transfer a portion of their energy to biological fluorescent molecules via Forster resonance energy transfer, or from direct ionization. The resulting optical signal, generated along the beam throughout its depth of interaction to the Bragg peak location, is weak but detectable.

### SUMMARY

A system for performing radiation treatment of a patient with a proton beam from a particle accelerator uses a high-sensitivity camera to capture dose images of patient surface, a video processor that integrates the dose images, beam-on detection apparatus, and apparatus to eliminate interference of room lighting. In embodiments, the system registers dose images to a surface model of the patient derived from stereo image pairs captured by a stereo camera. In particular embodiments, the surface model is registered to three-dimensional images of the patient from MRI or CT, and an integrated three-dimensional energy deposition map of the patient is prepared.

In one embodiment, a system for performing radiation treatment of a patient includes: a particle accelerator configured to provide a pulsed proton beam; beam-on detection apparatus configured for determining beam-on times, the beam-on times being when each pulse of the proton beam is provided by the particle accelerator; a high-sensitivity camera positioned to capture dose images of a surface of the patient exposed to the pulsed proton beam; a video processor configured to prepare integrated dose images of the surface of the patient from the dose images of the surface of the patient; and apparatus to eliminate interference of room lighting with the dose images.

In another embodiment, a method determines a radiation dosage map of a patient exposed to a therapeutic proton beam. The method includes: positioning the patient in a treatment zone; providing a therapeutic proton beam to the patient; imaging light generated by interaction of the therapeutic proton beam with a skin surface of the patient using a high sensitivity camera to form dose images; eliminating interference of room lighting with the dose images; and integrating the dose images to form integrated dose images.

### BRIEF DESCRIPTION OF THE DRAWINGS

FIG. 1 is a sketch illustrating an embodiment of a system for mapping radiation dose during proton-beam radiation treatment of a patient wherein image capture is triggered by detection of scattered radiation.

FIG. 2 is a sketch of another embodiment of a system for mapping radiation dose during proton-beam radiation treatment of a patient wherein image capture is triggered directly by a radiation detector. Features having the same reference number in FIGS. 1 and 2 are similar and have similar function.

FIG. 3 is a photograph made with an intensified camera of an intersection point of a proton beam and a piece of meat.

FIG. 4 represents cumulative exposure from a proton beam on the piece of meat of FIG. 3, the proton beam being swept in a pattern during a treatment session.

FIG. 5 is a flowchart illustrating a method of determining cumulative exposure during a treatment session of a patient from a proton beam.

### DETAILED DESCRIPTION OF THE EMBODIMENTS

We have found it desirable to quantitatively map intersections of a therapeutic proton beam with a skin surface of patients to be treated, and to do so in real-time while tracking patient and beam movements. For purposes of this document, the term “patients” includes both humans and animals, and the term “treatment” includes proton beam exposure of patients for research as well as for treatment of diseases such as cancer. By mapping beam-patient intersections, we can determine treatment dose profiles within each patient and map patient skin-surface exposure; this information is of use in dose and beam profile adjustment to preserve skin function while ensuring sufficient radiation is provided to destroy tumors within the patients.

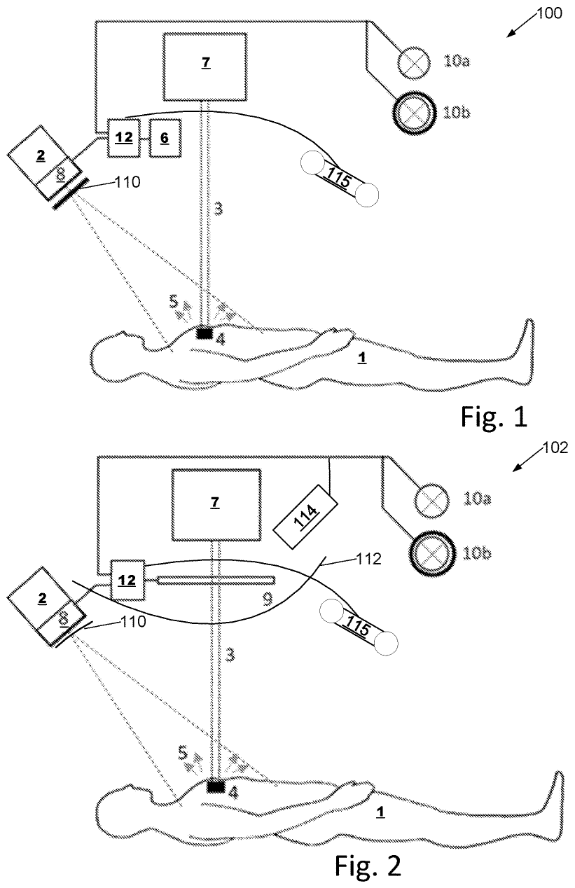

Systems 100, 102 (FIG. 1 and FIG. 2) for mapping radiation dose during proton-beam radiation treatment of a patient 1 include a high sensitivity camera 2. In an embodiment, high sensitivity camera 2 is an intensified CMOS camera. In alternative embodiments, an intensified CCD camera, an array of single photon avalanche photodiodes (SPAD), or another gateable two-dimensional, high sensitivity, visible-light detector is used as high sensitivity camera 2. In all systems, camera 2 is a high-sensitivity camera 2 positioned to image skin surface of patient 1. In a particular embodiment, high-sensitivity camera 2 is adapted to readout one image frame while integrating light for a following image frame, this overlap of integrating light and reading out images permits the high-sensitivity camera to image in low light levels. Further, overlap of image readout with integration for a following image frame allows avoidance of data loss between image frames and improved quantitative analysis because otherwise data regarding fast beam movement or skipping may be missed by the camera.

A tumor, not shown, is treated by a proton beam 3. The proton beam 3 intersects patient skin at an intersection point 4, typically a 0-5-millimeter-thick surface of patient of skin with underlying anatomy, which is subject to radiation dosage and damage from the proton beam 3.

Visible light 5, is emitted from the entrance dose surface or intersection point 4 of beam and patient, this light originates from scintillation, auto-fluorescence, and/or Cherenkov processes within the patient; light generated by entrance of the beam into the patient at the intersection point 4 is referred to herein as beam-patient intersection light. Visible light 5 passes to high sensitivity camera 2 through a gating and photon-intensifying unit 8 that is an image intensifier where high sensitivity camera 2 is an intensified CCD or CMOS camera, or avalanche photodiodes and gating electronics where high sensitivity camera 2 is a SPAD array. Images of the intersection of beam and patient from the camera 2 are captured in a video and control processor 12.

The proton beam 3 is directed at the patient 1, and in embodiments swept across portions of the patient 1, from an output nozzle 7 of a proton therapy beamline machine such as a cyclotron or cyclo-synchrotron particle accelerator. High sensitivity camera 2 is positioned to image the visible light 5 emitted from the beam-patient intersection point 4.

In embodiments, the proton beam is delivered as pulsed at rate that corresponds to an orbital or extraction frequency of cyclotron or cyclo-synchrotron particle accelerator.

Ordinary room lighting is far more intense than beam-patient intersection light. Were ordinary room lighting present when high-sensitivity camera 2 images beam-patient intersection light, the ordinary room lighting would swamp out the beam-patient intersection light in images.

In embodiments, high-speed gateable room lighting 10b is provided under control of video and control processor 12; such high-speed gateable room lighting may be formed from an array of light-emitting diodes of various colors selected such that light from the array appears white to eyes of patient 1. In these embodiments, video and control processor 12 is configured to turn off the room lighting 10b when the proton beam 3 is being emitted from output nozzle 7 to avoid interference of room lighting with high-sensitivity camera 2.

In alternative embodiments, ungated room lighting 10a is provided, however this ungated room lighting 10a is configured as an array of light-emitting diodes of selected specific lighting wavelengths, such as red and yellow or green light-emitting diodes that do not emit broadband light. Notch filters 110 are provided to block the selected specific lighting wavelengths emitted by the light-emitting diodes from high sensitivity camera 2, thereby preventing interference by light of the ungated room lighting with images of beam-patient intersection light. In this embodiment, the dose images of beam-patient intersection light are made by imaging visible light wavelengths other than the selected specific wavelengths provided by room lighting 10a. Further, in this embodiment it is not necessary that the camera be gated.

In alternative embodiments, video and control processor 12 is configured to capture a background image of patient 1 as illuminated by dim ungated room lighting 10a, to capture a sequence of dose images of the patient in beam-patient intersection light, and to subtract the background image of patient 1 from each image of the sequence of images of beam-patient intersection light to provide a corrected sequence of images of beam-patient intersection light.

For convenience herein, the images of beam-patient intersection light are referred to as Cherenkov images or dose images.

For purposes of this document, high-speed gateable room lighting controlled to be turned off when the proton beam is on, room lighting configured to use specific lighting wavelengths with filters to prevent these specific wavelengths from interfering with dose images, and a video and control processor configured to subtract a background image from dose images, are all considered apparatus to eliminate interference of room lighting with dose images.

The dose images provide a point of impact of beam on skin. A second point along the beam can be derived by placing a thin sheet of scintillator, which can serve as a fast response direct radiation detector 9 (FIG. 2), in the beam path from output port 7 to patient 1, and imaging that sheet of scintillator with a scintillator-viewing electronic camera 114. To prevent interference with the high-sensitivity camera 2, a thin sheet of black plastic 112 serves as a light shield to block light from the scintillator from reaching the patient and high-sensitivity camera 2. In an alternative embodiment, the second point along the beam is inferred from location of the output port 7 of the accelerator or location of beam-steering magnets in the beam path. The point of impact of beam on skin and the second point along the beam are used by video and control processor 12 to determine a beam vector at each point of impact of beam on skin.

In an embodiment, one or both of the scintillator camera images and the dose images are used to quantify and validate beam dimensions, and to record any intensity fluctuations of the beam that may alter dose rate or beam distribution.

In alternative embodiments, a device for mapping skin surface in real time is provided. In an embodiment, the device for mapping skin surface is a stereo imaging camera 115 sensitive to the specific lighting wavelengths of the room lighting is provided to capture stereo images of the patient 1 and to permit extraction of a patient surface model by the video and control processor 12 from the stereo images while avoiding interference with high sensitivity camera 2. In an alternative embodiment, a lidar unit using a laser and time-of-flight rangefinder is provided as a device for mapping skin surface, the lidar uses laser illumination at a wavelength blocked by a filter at high sensitivity camera 2 to prevent interference with high sensitivity camera 2. In another alternative embodiment, the patient skin surface is illuminated with structured light and a camera is used to obtain images from which a map of skin surface may be obtained.

In an embodiment the patient surface model derived from the device for mapping skin surface is a moving surface model adapted to record relative motion of patient 1 and proton beam 3.

In an embodiment, arrival of proton beam 3 is detected by an indirect triggering unit 6, such as a fast detector for scattered radiation from the proton beam.

In an alternative embodiment, arrival of proton beam 3 is detected by a direct triggering unit such as a fast radiation detector 9 positioned within the proton beam 3.

In another alternative embodiment, a “beam on” trigger signal is used to determine arrival of the proton beam 3, this beam-on signal being provided by the particle accelerator (typically a cyclotron or a cyclo-synchrotron) used to provide the proton beam at output nozzle 7.

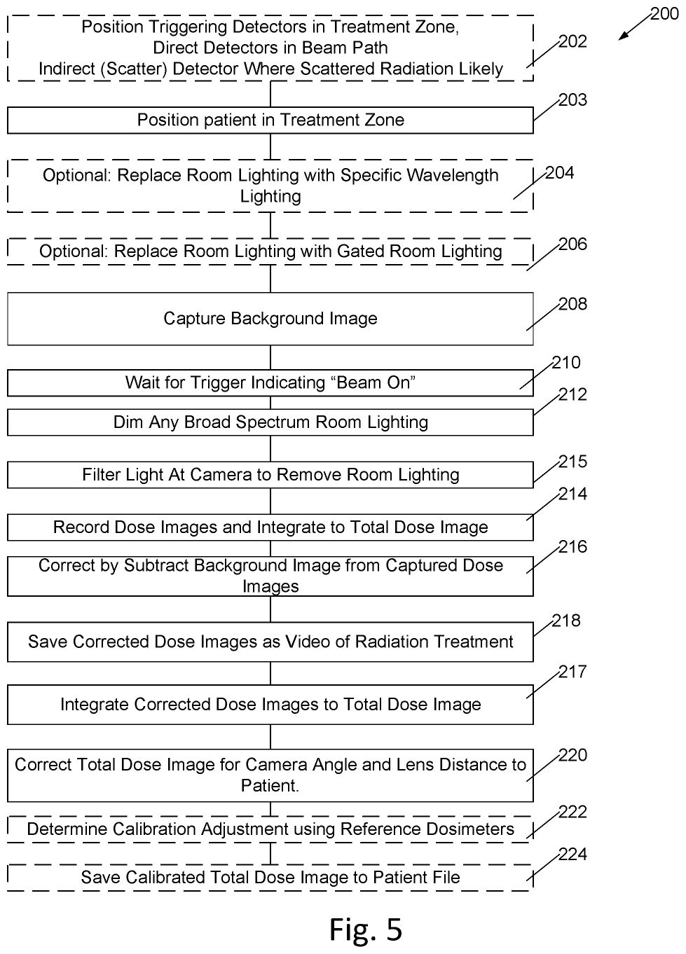

Systems 100, 102 (FIGS. 1 and 2) operate according to the method 200 illustrated with the flowchart of FIG. 5. If a “beam on” signal from the particle accelerator is unavailable, triggering detectors are positioned 202 in a treatment zone, the treatment zone being a space between output nozzle 7 and a radiation shield (not shown) that absorbs radiation that penetrates the patient. The patient 1 is positioned 203, typically on a couch that is mounted on a robotic arm, a gurney, or in a chair, within the treatment zone. In an embodiment, the triggering detectors are direct radiation detectors 9 placed in a beam path from output nozzle 7 to patient 1, and in another embodiment the triggering detectors are indirect or scatter-based detectors 6 positioned where scattered radiation is likely when the beam is on.

Next, ordinary room lighting (if any) is turned off, being replaced 206 with fast-acting gateable lighting 10b that can be rapidly turned on or off, replaced 204 with lighting 10a of specific defined wavelengths, or with lighting that is both of defined wavelengths and gateable. A background image 208 is also captured.

The system waits 210 for a “beam-on” trigger signal from the particle accelerator, direct radiation detectors 9, or from the indirect or scatter-based detectors 6. Once the “beam-on” trigger signal is received, any broad-spectrum gateable room lighting 10b is dimmed 212, and, if room lighting is of specific defined wavelengths 10a, light of those specific defined wavelengths is prevented 215 from reaching high-sensitivity camera 2 by appropriate filters; the high-sensitivity camera records 214 dose images in light of wavelengths not used by specific-wavelength room lighting 10a that are emitted from the intersection point 4.

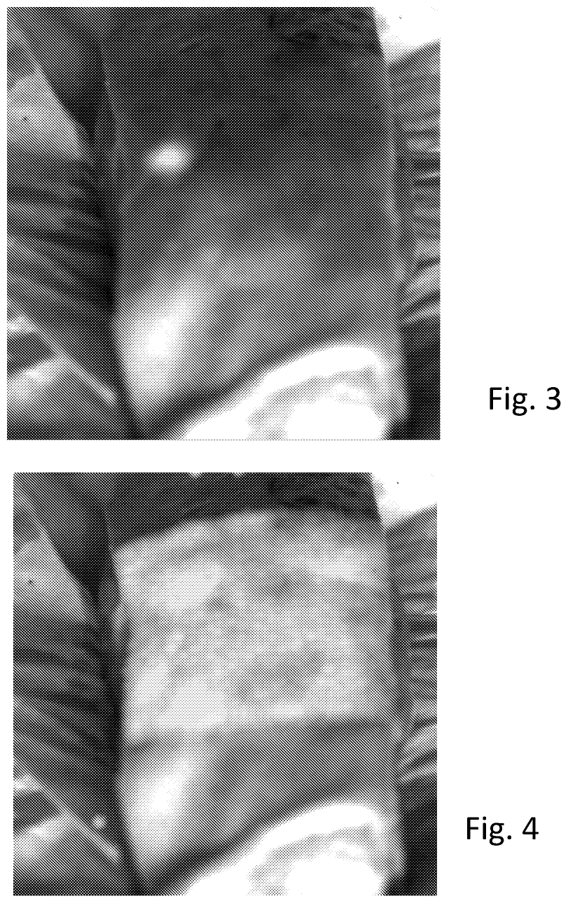

The dose images are then corrected by subtracting 216 the background image from the dose images; the corrected dose images, such as the dose image illustrated in FIG. 3 where the bright spot represents light emitted from interaction of the proton beam with tissue, are saved 218 in a motion picture of radiation dose received by the patient 1. The dose images are also integrated 217 to provide a total dose image as illustrated by the bright rectangle in FIG. 4. The total dose image is corrected 220 for camera angle and distance to the subject, the corrected total dose image is adjusted as necessary for calibration 222 and recorded 224 in the patient file as a calibrated image of received radiation dose. The corrected total radiation dose is compared to limits and radiation treatment is stopped on reaching a limit dose.

In embodiments using room lighting 10a of specific wavelengths that are excluded from capture by high-sensitivity camera 2 when high-sensitivity camera 2 captures dose images, an additional stereo camera 115 captures stereo image pairs of patient 1 as illuminated by the room lighting 10a. In these embodiments, a surface model of patient 1 is derived by video and control processor 12 from the stereo image pairs and updated sufficiently fast as to show patient movements. In an alternative embodiment, the surface model is derived from another device for mapping patient skin surface such as an infrared lidar or a lidar operating at another wavelength that is blocked by a filter at high-sensitivity camera 2 to prevent interference with high-sensitivity camera 2. Patient movement may include patient movements induced by a patient rotating turntable or a patient repositioning machine such as a robotic arm upon which the patient is seated in a couch, these movements may be intended to reduce skin exposure during irradiation of a tumor. In this embodiment, corrected dose images are captured and registered to the surface model of the patient at the rate sufficient to show movement of the patient; these registered dose images are then integrated to show total dose received by the patient throughout a treatment session. Overlapping frame readout of one image with photosensor integration in camera 2 assists with generating quantitative total dose maps by avoiding dropouts that may miss rapid movements of patient or beam.

In an embodiment, the surface model of the patient determined from the stereo image pairs is registered by the video and control processor to a three-dimensional voxel-based image of the patient such as may be obtained with a computed tomography (CT) scanner or magnetic resonance imaging (MRI) scanner. The dose images are used to determine quantified beam vectors within the three-dimensional voxel-based image of the patient, and a beam energy-deposition model is applied to the beam vectors to determine instantaneous energy deposition within the three-dimensional voxel-based image of the patient. The instantaneous energy deposition is then integrated to prepare an integrated three-dimensional energy deposition map of the treatment session showing where within the patient energy was deposited through each treatment session; the energy deposition map may be used to estimate treatment effectiveness or dose-volume agreement with treatment plans.

In an alternative embodiment, thin sheets of a calibrated scintillator material are positioned within the treatment zone above or on the patient prior to exposing the patient to the proton beam. In this embodiment, the proton beam induces scintillation in the thin sheets of scintillator material and a second high-sensitivity camera is positioned to form scintillator dose images of the thin sheets of scintillator material. In this embodiment, the integrated dose images are calibrated to the scintillator dose images.

In embodiments where beam vectors are derived from locations of the beam intersection of skin and a second point along the beam, video and control processor 12 may register the skin surface model to a voxel-based three-dimensional model, the model including patient density such as may be obtained by pre-treatment X-ray computed tomography (CT) scans, then apply a radiation absorption model to determine an effective dose at each voxel of the three-dimensional model. The effective dose at each voxel may then be compared to a treatment plan.

### Combinations

The features and steps herein described can be combined in a multitude of ways. Among combinations anticipated by the inventors are:

A system designated A for performing radiation treatment of a patient includes a particle accelerator configured to provide a pulsed proton beam; beam-on detection apparatus configured for determining beam-on times, the beam-on times being when each pulse of the proton beam is provided by the particle accelerator; a high-sensitivity camera positioned to capture dose images of a surface of the patient exposed to the pulsed proton beam where the proton beam intersects surface of the patient; a video processor configured to prepare integrated dose images of the surface of the patient from the dose images of the surface of the patient; and apparatus to eliminate interference of room lighting with the dose images.

A system designated AA including the system designated A wherein the apparatus to eliminate interference of room lighting with the dose images comprises room lighting configured to emit specific room lighting wavelengths and filters configured to block the specific room lighting wavelengths from the high sensitivity camera, and further comprising a device for determining a surface model of the patient; the video processor being configured to register the dose images to the surface model of the patient; wherein the high-sensitivity camera is configured to image wavelengths of visible light other than the specific room lighting wavelengths

A system designated AB including the system designated A or AA wherein the device for determining a surface model of the patient comprises a stereo camera sensitive to the specific room lighting wavelengths and a processor configured to extract a surface model from stereo image pairs captured by the stereo camera.

A system designated AC including the system designated A, AA, or AB wherein the video processor is configured to register the surface model of the patient to a three-dimensional voxel-based model of the patient, to use the dose images to determine beam vectors within the three-dimensional image of the patient, to apply a beam energy-deposition model to the dose images, and to prepare an integrated three-dimensional energy deposition map of the patient.

A system designated AD including the system designated A, AA, AB, or AC wherein the three-dimensional voxel-based model of the patient is generated by a computed X-Ray tomography (CT) system or a nuclear magnetic resonance imaging (MRI) system.

A system designated AE including the system designated A, AA, AB, AC, or AD wherein the high-sensitivity camera is configured to read out a first frame while photosensors of the high-sensitivity camera integrate light for a second frame.

A method designated B of determining a radiation dosage map of a patient exposed to a therapeutic proton beam includes positioning the patient in a treatment zone; providing a therapeutic proton beam to the patient; imaging light generated by interaction of the therapeutic proton beam with a skin surface of the patient using a high sensitivity camera to form dose images; eliminating interference of room lighting with the dose images; and integrating the dose images to form integrated dose images.

A method designated BA including the method designated B further including generating a surface model of the patient; and registering the integrated dose images to the surface model.

A method designated BB including the method designated BA where generating a surface model of the patient is performed by capturing stereo image pairs of the patient and extracting a surface model from the stereo image pairs.

A method designated BC including the method designated BA where generating a surface model of the patient is performed with an infrared lidar.

A method designated BD including the method designated BA, BB, or BC further including registering the surface model to a three-dimensional model of the patient; determining beam vectors where the therapeutic proton beam intersects the patient; and using an absorption model to determine radiation dose at voxels of the three-dimensional model of the patient.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A system for performing radiation treatment of a patient comprises:
a particle accelerator configured to provide a pulsed proton beam;
beam-on detection apparatus configured for determining beam-on times by detecting scattered radiation, the beam-on times being when each pulse of the proton beam is provided by the particle accelerator;
a high-sensitivity camera positioned to capture dose images of a surface of the patient exposed to the pulsed proton beam;
a video processor configured to prepare integrated dose images of the surface of the patient from the dose images of the surface of the patient; and
apparatus to eliminate interference of room lighting with the dose images.

2. The system of claim 1, wherein the apparatus to eliminate interference of room lighting with the dose images comprises room lighting configured to emit specific room lighting wavelengths and filters configured to block the specific room lighting wavelengths from the high sensitivity camera, and further comprising a device for determining a surface model of the patient; the video processor being configured to register the dose images to the surface model of the patient; wherein the high-sensitivity camera is configured to image wavelengths of visible light other than the specific room lighting wavelengths

3. The system of claim 2, wherein the device for determining a surface model of the patient comprises a stereo camera sensitive to the specific room lighting wavelengths and a processor configured to extract a surface model from stereo image pairs captured by the stereo camera.

4. The system of claim 2, wherein the video processor is configured to register the surface model of the patient to a three-dimensional voxel-based model of the patient, to use the dose images to determine beam vectors within the three-dimensional image of the patient, to apply a beam energy-deposition model to the dose images, and to prepare an integrated three-dimensional energy deposition map of the patient.

5. The system of claim 4, wherein the three-dimensional voxel-based model of the patient is generated by a computed X-Ray tomography (CT) system or a nuclear magnetic resonance imaging (MM) system.

6. The system of claim 1, wherein the high-sensitivity camera is configured to read out a first frame while photosensors of the high-sensitivity camera integrate light for a second frame.

7. A method of determining a radiation dosage map of a patient exposed to a therapeutic proton beam, the method comprising:
positioning the patient in a treatment zone;
providing a therapeutic proton beam to the patient;
imaging light generated by interaction of the therapeutic proton beam with a skin surface of the patient using a high sensitivity camera to form dose images;
eliminating interference of room lighting with the dose images; and
integrating the dose images to form integrated dose images.

8. The method of claim 7, further comprising:
generating a surface model of the patient; and
registering the integrated dose images to the surface model.

9. The method of claim 8, where generating a surface model of the patient is performed by capturing stereo image pairs of the patient and extracting a surface model from the stereo image pairs.

10. The method of claim 8, where generating a surface model of the patient is performed with an infrared lidar.

11. The method of claim 8, further comprising:
registering the surface model to a three-dimensional model of the patient;
determining beam vectors where the therapeutic proton beam intersects the patient; and
using an absorption model to determine radiation dose at voxels of the three-dimensional model of the patient.

12. The system of claim 2, wherein the high-sensitivity camera is configured to read out a first frame while photosensors of the high-sensitivity camera integrate light for a second frame.

13. The system of claim 3, wherein the high-sensitivity camera is configured to read out a first frame while photosensors of the high-sensitivity camera integrate light for a second frame.

14. The system of claim 4, wherein the high-sensitivity camera is configured to read out a first frame while photosensors of the high-sensitivity camera integrate light for a second frame.

15. The system of claim 5, wherein the high-sensitivity camera is configured to read out a first frame while photosensors of the high-sensitivity camera integrate light for a second frame.

16. The method of claim 9, further comprising:
registering the surface model to a three-dimensional model of the patient;
determining beam vectors where the therapeutic proton beam intersects the patient; and
using an absorption model to determine radiation dose at voxels of the three-dimensional model of the patient.

17. The method of claim 10, further comprising:
registering the surface model to a three-dimensional model of the patient;
determining beam vectors where the therapeutic proton beam intersects the patient; and
using an absorption model to determine radiation dose at voxels of the three-dimensional model of the patient.
